(19) United States
(12) Patent Application Publication
(10) Pub. No.: US 2023/0285089 A1
(43) Pub. Date: Sep. 14, 2023

(54) Auxiliary Apparatus for Surgical Operations

(71) Applicant: MEDICS SRL, Torino (IT)

(72) Inventors: Diego Manfrin, Vado Ligure (IT); Andrea Bellin, Torino (IT); Leandro Gianmaria BASSO, Torino (IT); Andrea ANDOLFI, Moncalieri (IT); Giuseppe ISU, Torino (IT)

(21) Appl. No.: 18/019,266

(22) PCT Filed: Aug. 5, 2021
(86) PCT No.: PCT/IB2021/057212

§ 371 Date: Feb. 2, 2023

(30) Foreign Application Priority Data
Aug. 6, 2020 (IT) 102020000019417

(51) Int. Cl. A61B 34/00; G06T 17/00; G06T 19/00; A61B 50/13; A61B 90/00; A61B 34/37; G16H 40/63; G16H 30/40

(52) U.S. Cl. CPC A61B 34/25; G06T 17/00; G06T 19/00; A61B 50/13; A61B 90/361; A61B 34/37; G16H 40/63; G16H 30/40; G06T 2210/41; G06T 2200/24; A61B 2090/367; A61B 2034/105

(57) ABSTRACT

An auxiliary apparatus for surgical operations, comprising a structure which supports a processing unit programmed to manage a virtual three-dimensional model of an organ to be operated on. A monitor visualizes the virtual three-dimensional model. A contactless sensor detects the movements of a hand and is connected functionally to the processing unit in order to virtually manipulate the virtual three-dimensional model as a function of the movements of the hand. The processing unit displays on the monitor an image that represents the hand, is adapted to follow the movements of the hand in the detection field of the sensor, and is variable at least as a function of specific movements of the hand that correspond to respective manipulation commands and/or operating commands.

The present invention relates to an auxiliary apparatus for surgical operations.

As is known, surgical operations in an operating room currently can be performed according to various operating methods, among which mention is made of:
so called “open surgery”, in which the surgeon operates personally on the patient with the aid of manual instruments and views the operating field directly through an incision provided in the body of the patient;
so called “endoscopic surgery” (laparoscopic or thoracoscopic), in which the surgeon operates personally on the patient with the aid of manual instruments and views the operating field by means of a viewer which reproduces the video stream of an endoscopic video camera inserted through an incision provided in the body of the patient;
so called “robotic surgery”, in which the operation is performed by a robot which is controlled remotely by a surgeon, who has a computerized control console located in the operating room. The control console is generally provided with a three-dimensional viewer, which receives images from one or more stereo video cameras arranged so as to view the operating field, and with controls which reproduce the handle of the surgical instruments used by the robot (forceps, scissors, dissectors, etc.).

In the case of robotic surgery, it is known from WO2019137895 to alternate, arrange side-by-side and/or superimpose on the video stream generated by the stereoscopic video camera a virtual three-dimensional image of the organ to be operated on, which can be shifted and oriented by virtue of manipulation means, such as a 3D mouse, a joystick, etc., driven by an auxiliary operator.

The virtual three-dimensional image can be reconstructed starting from two-dimensional images generated by computerized tomography or magnetic resonance.

The system described in WO2019137895 assists the surgeon in identifying the contours of the anatomical parts viewed by the stereoscopic video camera and consequently in reaching the points where to operate with the instruments, but it has the limitation that it is conceived only for robotic surgery and necessarily requires the surgeon to be assisted by an operator assigned to manipulating the virtual three-dimensional image.

In view of the above, the aim of the present invention is to provide an auxiliary apparatus for surgical operations which is adapted to display on a monitor a virtual three-dimensional image of the organ to be operated on, which can also be applied to manual operating methods and allows the surgeon to move and/or orient the virtual three-dimensional image on his own, therefore without the aid of assistants, in a practical and intuitive manner and in full compliance with the hygiene protocols to be followed in operating rooms.

This aim and other objects which will become more apparent from the continuation of the description are achieved by an auxiliary apparatus for surgical operations having the characteristics presented in claim 1, while the dependent claims define other advantageous, albeit secondary, characteristics of the invention.

The invention is now described in greater detail with reference to a preferred but not exclusive embodiment thereof, illustrated by way of non-limiting example in the accompanying drawings, wherein:

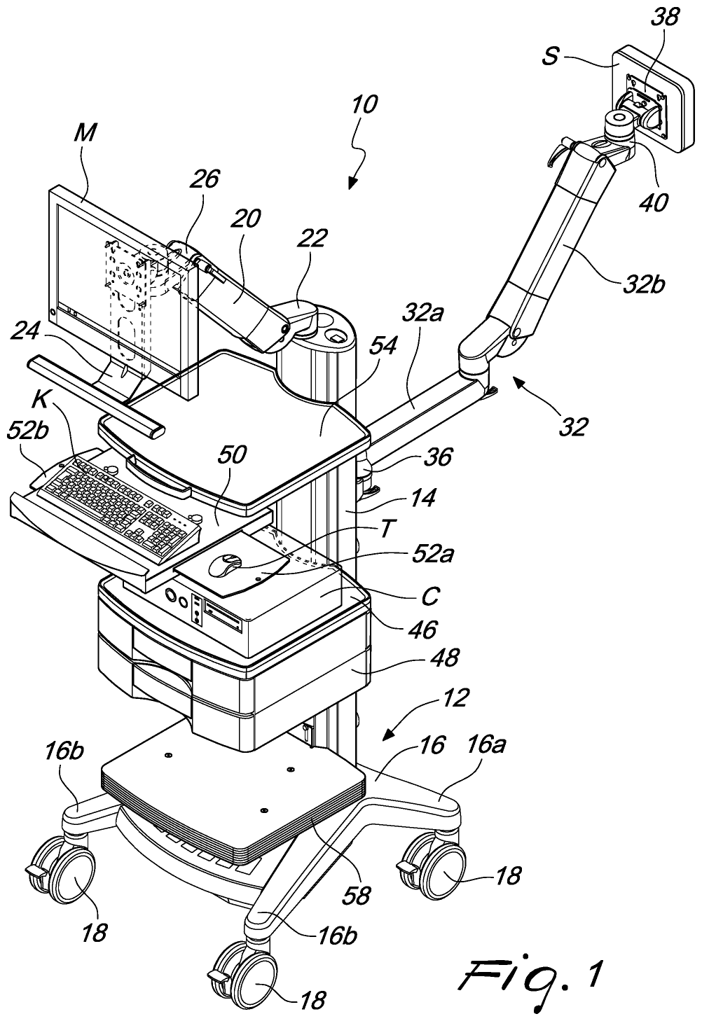

FIG. 1 is a perspective view of an auxiliary apparatus according to the invention;

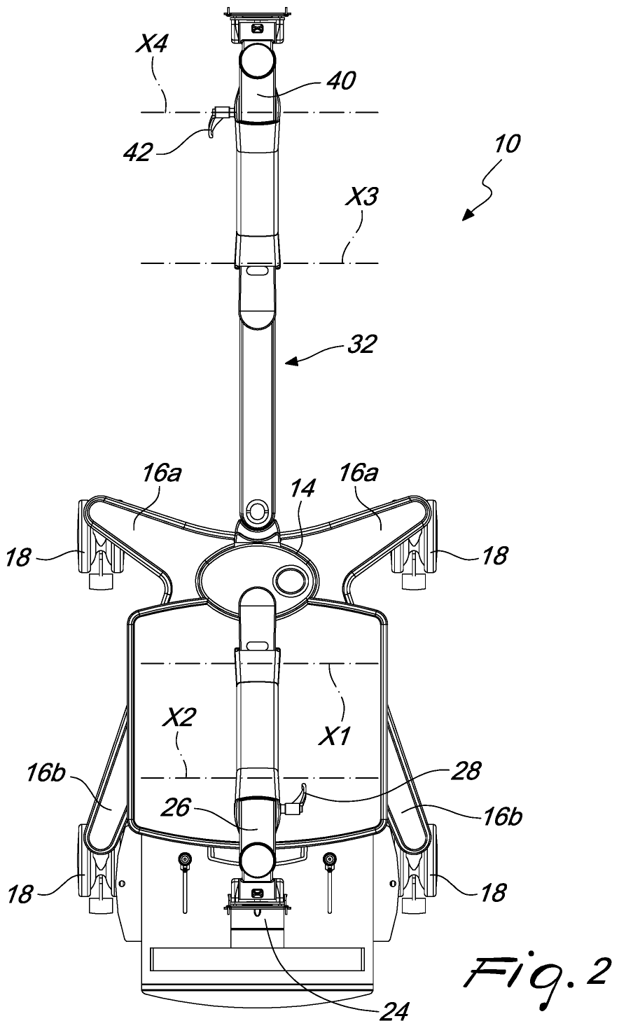

FIG. 2 is a lateral elevation view of the auxiliary apparatus of FIG. 1,

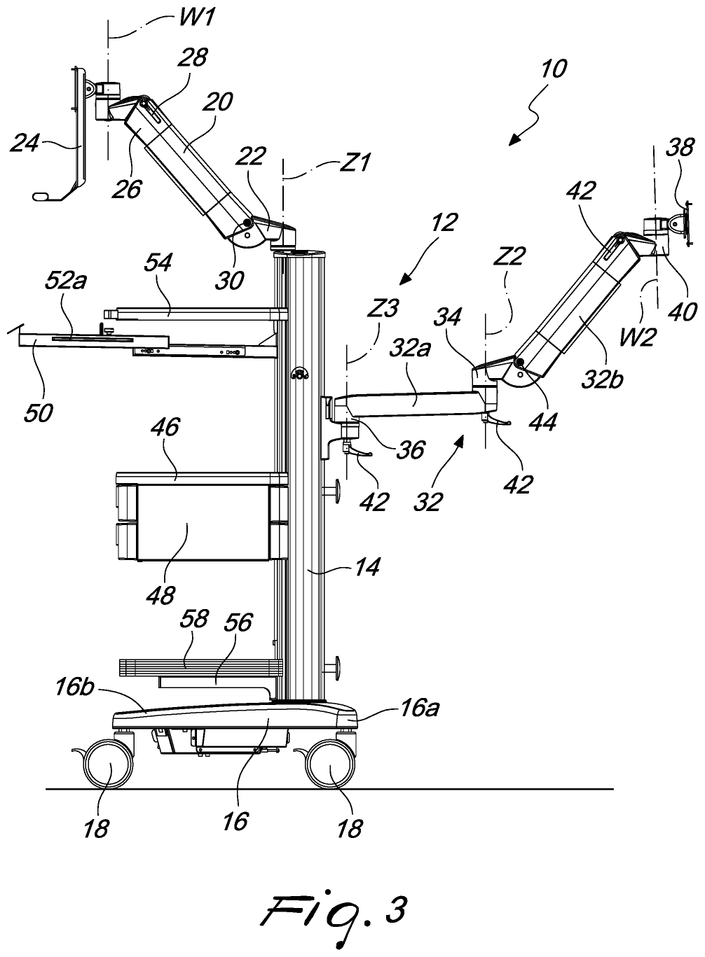

FIG. 3 is a plan view of the auxiliary apparatus of FIG. 1;

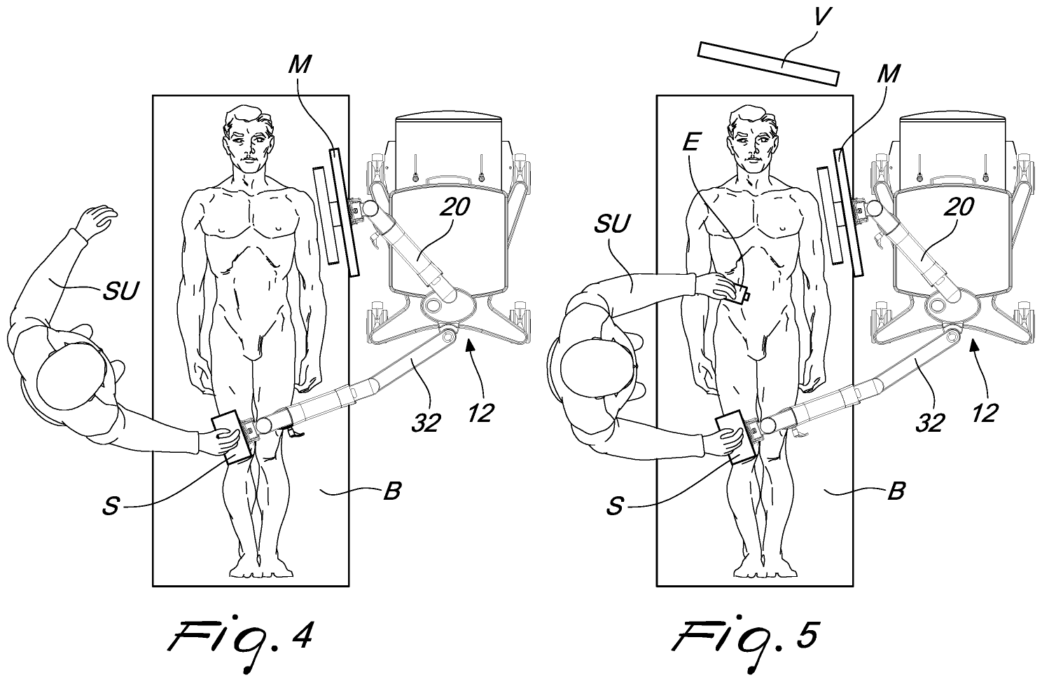

FIG. 4 is a plan view of the auxiliary apparatus of FIG. 1 in an operating room in a first mode of use;

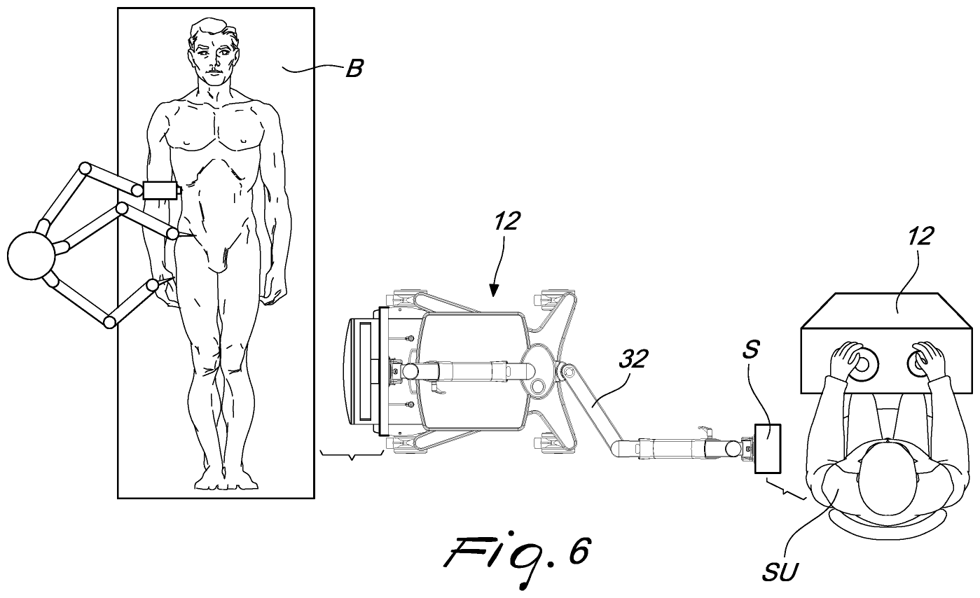

FIG. 5 is a plan view of the auxiliary apparatus of FIG. 1 in an operating room, in a second mode of use;

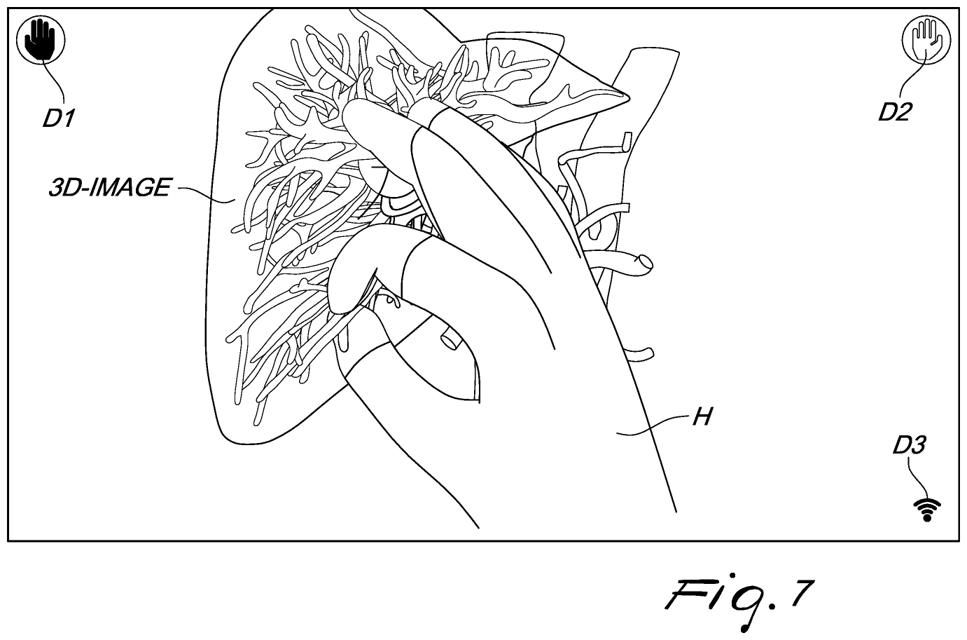

FIG. 6 is a plan view of the auxiliary apparatus of FIG. 1 in an operating room, in a third mode of use;

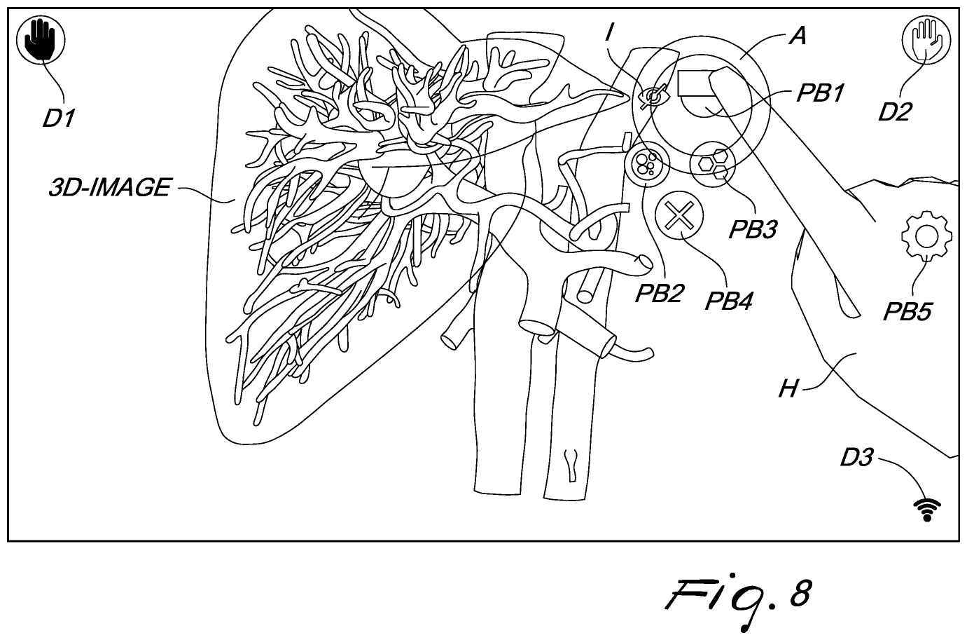

FIG. 7 is a view of a screen of the auxiliary apparatus according to the invention in a first step of use;

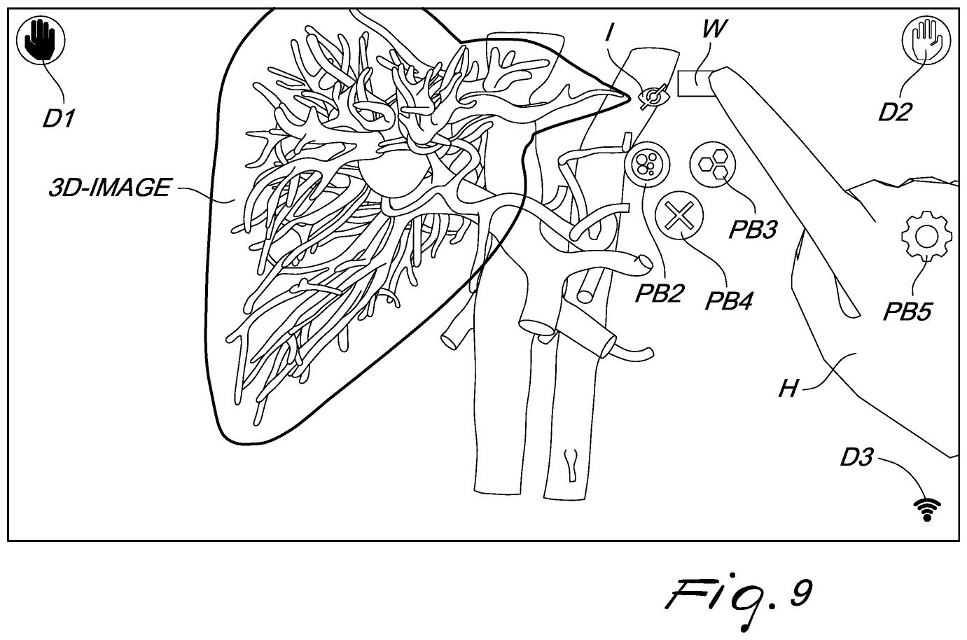

FIG. 8 is a view of a screen of the auxiliary apparatus according to the invention in a second step of use;

FIG. 9 is a view of a screen of the auxiliary apparatus according to the invention in a third step of use.

With reference to the figures, an auxiliary apparatus for surgical operations according to the invention, designated generally by the reference numeral 10, comprises a supporting structure, advantageously a wheeled structure or cart 12, which supports
a processing unit C programmed for the management of a virtual three-dimensional model 3D-IMAGE (FIGS. 7-9) of an organ to be operated on,
a monitor M functionally connected to the processing unit C in order to visualize the virtual three-dimensional model 3D-IMAGE,
a contactless sensor S adapted to detect the movements of a hand of the surgeon and connected functionally to the processing unit C in order to manipulate virtually the virtual three-dimensional model 3D-IMAGE as a function of said movements of the hand,
the processing unit C being furthermore programmed to display on the monitor M an image H (FIGS. 7-9) which represents the hand of the surgeon, is adapted to follow the movements of the hand in the detection field of the sensor S and is variable at least as a function of specific movements of the hand that correspond to respective manipulation commands and/or operating commands.

The electronic devices supported on the cart 12 have been removed in FIGS. 2 and 3 for greater clarity of illustration.

In a manner known per se, the virtual three-dimensional model 3D-IMAGE is advantageously reconstructed starting from two-dimensional images generated by computerized tomography or magnetic resonance of an organ to be operated on of the specific patient.

FIGS. 7-9 show, by way of example, three respective screens which can be displayed on the monitor M during the use of the auxiliary apparatus according to the invention.

In the example shown in FIG. 7, the image H shows in stylized form the hand of the surgeon in the gesture of grasping the virtual three-dimensional model 3D-IMAGE of the organ to be operated on, which in this example is a liver, for example in order to rotate it. Other gestures of the hand of the surgeon can be translated into respective manipulation commands which are adapted for example to shift or scale the virtual three-dimensional model 3D-IMAGE. At each one of these manipulation commands, the image H of the hand reproduced on the monitor M can assume a respective position which advantageously is evocative of the activated manipulation command.

In the absence of specific gestures adapted to activate respective manipulation commands, the image H of the hand reproduced on the monitor M can advantageously assume a position which is fixed and neutral with respect to manipulation actions. Advantageously, such neutral position can optionally be used by the user to perform indications on the monitor, in particular on the three-dimensional virtual model or on any video stream of a surgical video camera inserted as background, as will be shown in detail hereinafter.

In a different embodiment, the image H of the hand reproduced on the monitor M, instead of varying only upon the execution of specific gestures which correspond to respective manipulation commands or operating commands, may replicate more faithfully the movements of the hand of the surgeon and, in extreme cases, follow in a substantially continuous manner and in real time all the movements of the hand.

The choice among the embodiments described above can be determined by the type of sensor used, by the detection resolution of the sensor, by the processing capacity of the processing unit C, by programming choices and by other similar factors, as well as by the preferences of the surgeon and, in this last case, may also constitute one of the parameters that can be set by the user.

Advantageously, a specific gesture, for example the rotation of the hand with the palm facing upward, allows the surgeon to activate a menu to modify settings and/or execute operating commands.

The menu is preferably organized with one or more submenus which group different components of the virtual three-dimensional model 3D-IMAGE on the basis of anatomical type.

In FIG. 8, the image H represents in stylized form the hand of the surgeon in the gesture of activating preset functions on a specific component of the virtual three-dimensional model 3D-IMAGE, by virtual pressing of one of a series of virtual pushbuttons PB1, PB2, PB3, PB4, PB5 displayed on the monitor M. In particular, the virtual pushbuttons PB1, PB2, PB3 may render respective components of the virtual three-dimensional model 3D-IMAGE, which are initially shown opaque, semitransparent (for example upon first pressing) or hidden (for example upon second pressing). The pressing of a virtual pushbutton is advantageously confirmed visually by a ring A which surrounds said virtual pushbutton, while the chosen function (in the example above, semitransparent or hidden visualization) is represented by an icon I to the side of the virtual pushbutton.

Advantageously, as shown in FIG. 9, when the index finger of the image H of the hand approaches the virtual pushbutton that corresponds to a certain component of the virtual three-dimensional model 3D-IMAGE, the edge E of said component is highlighted and preferably a descriptive caption W which shows the name of the portion appears at the virtual pushbutton.

Other informational icons, such as D1, D2, D3, can be displayed on the screen.

In the example of FIGS. 8 and 9, the virtual pushbutton PB4 closes the menu, while the virtual pushbutton PB5 allows entrance into the settings.

The processing unit C is preferably provided with a video card capable of receiving the video stream of a surgical video camera, for example an endoscopic video camera or a stereoscopic video camera, and is programmed to reproduce on the monitor M said video stream in the background, instead of a neutral background such as the one shown in FIGS. 7-9. This function preferably constitutes one of the settings that can be selected by the surgeon by means of the menu.

Advantageously, the video stream generated in output by the software has various formats so as to be able to adapt to the various systems and configurations with which it is associated, in particular 2D output or stereoscopic 3D output of what is called the “Split Channel” or “Dual Channel” type.

With particular reference now to FIGS. 1-3, in the embodiment described herein, illustrated by way of example, the cart 12 comprises a post 14 which rises from a base 16 which is mounted on four casters 18 which are arranged at the vertices of a rectangle. In particular, the base 16 is composed of two rear feet 16a and two front feet 16b in a star-like configuration, and the casters 18 are fixed to the free ends of the feet. The rear feet 16a are shorter than the front feet 16b, so that the post 14 is not arranged centrally with respect to the rectangle defined by the casters 18 but is axially offset toward the rear side of the base 16.

Advantageously, the monitor M is connected to the upper end of the post 14 by means of a first articulated arm 20.

In the example described herein, the first articulated arm 20 has an end which is connected to the upper end of the post 14 by means of a first two-axis joint 22. The first two-axis joint 22 allows rotation of the first articulated arm 20 about a first horizontal axis X1 and a first vertical axis Z1. The monitor M is fixed to a first bracket 24, which is connected to the opposite end of the first articulated arm 20 by means of a second two-axis joint 26. The second two-axis joint 26 allows a rotation of the monitor M about a second horizontal axis X2 and a first orientable axis W1 which lies on a vertical plane which contains the axis of the first articulated arm 20.

The positions of the joints 22, 26 about the respective axes can be locked by tightening levers 28 or bolts 30.

Advantageously, the sensor S is connected to the post 14, at an intermediate height thereof, by means of a second articulated arm 32.

In the example described herein, the second articulated arm 32 has a substantially horizontal proximal portion 32a and a distal portion 32b which is connected to the proximal portion by means of a third two-axis joint 34. The third two-axis joint 34 allows the distal portion 32b to rotate with respect to the proximal portion 32a about a third horizontal axis X3 and about a second vertical axis Z2.

The proximal portion 32a is connected to the post 14 by means of a hinge with a vertical axis 36, which allows rotating the proximal portion 32a about a third vertical axis Z3.

The sensor S is fixed to a second bracket 38, which is connected to the free end of the distal portion 32b by means of a fourth two-axis joint 40. The fourth two-axis joint 40 allows rotating the sensor S about a fourth horizontal axis X4 and a second orientable axis W2 which lies on a vertical plane which contains the axis of the distal portion 32b.

In this case also, the positions of the joints 34, 40 and of the hinge 36 about the respective axes can be locked by tightening levers 42 or bolts 44.

Preferably, the second articulated arm 32 is fixed to the rear side of the post 14.

Advantageously, the processing unit C is supported on a first fixed shelf 46 which is connected at an adjustable intermediate height of the post 14. Preferably, the first fixed shelf 46 is mounted on the front side of the post 14 so as not to interfere with the second articulated arm 32 and help to balance the weight thereof, especially when it is in the fully extended configuration.

Advantageously, the first fixed shelf 46 incorporates a set of drawers 48 for storing medical instruments.

A keyboard K, functionally connected to the control unit C, is advantageously supported on an extractable shelf 50 which is fixed to the post 14 on the front side thereof, above the first fixed shelf 46.

Preferably, the extractable shelf 50 is provided with at least one platform which can be extracted laterally for a mouse T, preferably two platforms 52a, 52b which are inserted at the respective opposite sides of the extractable shelf 50.

A second fixed shelf 54 is advantageously fixed to the post 14 on the front side thereof, directly above the extractable shelf 50.

A ledge 56 fixed on the front side of the post 14, directly above the base 16, supports counterweights 58 which are adapted to balance the weight of the second articulated arm 32.

The programming of the processing unit C is not delved into herein since it is within the normal knowledge of the person skilled in the art and is beyond the scope of the aim and objects of the present invention.

In general terms, the virtual three-dimensional image 3D-IMAGE can be stored in a memory block which is integrated in the processing unit C or in an external storage unit which can be connected to the processing unit C or also in a remote server which can be accessed by the processing unit via a data communications network.

In use, the auxiliary apparatus 10 can be arranged differently inside the operating room depending on the mode with which the surgical operation is performed.

In particular, with reference to FIG. 4, in the case of “open surgery” the cart 12 is positioned proximate to the operating bed B, with the first articulated arm 20 oriented so as to direct the monitor M toward the surgeon and the second articulated arm 32 oriented so as to have the sensor S in a position that is conveniently accessible by the surgeon SU with one hand.

With reference to FIG. 5, in the case of so called “endoscopic surgery” the first articulated arm 20 is oriented so as to arrange the monitor M to the side of the screen V which reproduces the video stream of the endoscopic video camera E, and the second articulated arm 32 is oriented so as to have the sensor S in a position that can be accessed conveniently by the surgeon SU with one hand.

With reference to FIG. 6, in the case of so called “robotic surgery” the cart 12 is positioned to the side of the control console P, with the second articulated arm 32 oriented so as to have the sensor S in a position that can be accessed conveniently by the surgeon SU with the hand, without the surgeon needing to move away from the control console P. In this case, the virtual three-dimensional image 3D-IMAGE can be advantageously sent to the control console P as an alternative, as an accompaniment, or superimposed with respect to the video stream sent by the stereoscopic video camera ST, in a manner similar to what is described in WO2019137895.

Once positioned in the most convenient manner depending on the operating mode, the surgeon, before sanitizing himself, turns on the processing unit C and starts the software for the management of the virtual three-dimensional models 3D-IMAGE.

Advantageously, the surgeon can perform login by means of his own user credentials, so that the software shows all the virtual three-dimensional models associated with him. At this point, the surgeon selects the virtual three-dimensional model related to the patient on which he must operate, which is loaded and displayed on the monitor M.

Once the virtual three-dimensional model has been loaded, the surgeon can sanitize himself and begin the surgical operation, using hand gestures to interact with the virtual three-dimensional model by means of the sensor S, without touching anything, so as to prevent any risk of contaminating the sterilized gloves normally used to perform surgical operations.

In practice it has been demonstrated that the auxiliary apparatus according to the invention achieves fully the intended aim and objects, since it can be applied both to manual operating methods and to robotic operating methods, and allows the surgeon to move and/or orient the virtual three-dimensional image on his own without the aid of assistants, in a practical and intuitive manner and without the risk of contaminations, therefore in full compliance with the hygiene protocols to be followed in operating rooms.

A preferred embodiment of the invention has been described, but of course the person skilled in the art may apply various modifications and variations, all of which are within the scope of the claims.

In particular, depending on the applications, the supporting structure can be provided differently from what has been described and illustrated by way of preferential example. For example, the shape of the articulated arms, particularly the number of joints and the orientation of the rotation axes, may be varied according to the requirements.

Furthermore, although the fixing of the monitor and of the sensor to respective articulated arms renders the auxiliary apparatus according to the invention particularly versatile in positioning, in some cases, depending on the spaces within the operating room, the monitor and/or sensor might be simply rested on respective shelves of the supporting structure.

The supporting structure itself might be fixed instead of movable on casters, and instead of having a post-like structure it might be configured differently, for example as a set of shelves.

The structure of the menu of the software that manages the virtual three-dimensional models also may be modified according to the specific applications, for example with pulldown submenus and the like.

The disclosures in Italian Patent Application no. 102020000019417, and in International Patent Application No. PCT/IB2021/057212, from which this application claims priority, are both incorporated herein by reference.

1-18. (canceled)

19. An auxiliary apparatus for surgical operations, comprising a structure which supports:
a processing unit programmed for management of a virtual three-dimensional model of an organ to be operated on,
a monitor functionally connected to said processing unit in order to visualize said virtual three-dimensional model,
a contactless sensor adapted to detect movements of a hand and connected functionally to said processing unit in order to virtually manipulate said virtual three-dimensional model as a function of said movements of the hand,
said processing unit being programmed to display on said monitor an image that represents said hand, which is adapted to follow the movements of the hand in a detection field of the sensor and is variable at least as a function of specific movements of the hand that correspond to respective manipulation commands and/or operating commands.

20. The auxiliary apparatus according to claim 19, wherein said virtual three-dimensional model is reconstructed starting from two-dimensional images generated by computerized tomography or magnetic resonance of an organ to be operated on of a specific patient.

21. The auxiliary apparatus according to claim 19, wherein in the absence of specific gestures adapted to activate respective manipulation commands, an image of the hand assumes a position which is fixed and neutral with respect to manipulation actions, and optionally can be used by a user to perform indications on said monitor.

22. The auxiliary apparatus according to claim 21, wherein said image replicates the movements of the hand in a substantially continuous manner and in real time.

23. The auxiliary apparatus according to claim 19, wherein said processing unit is programmed to interpret a specific gesture of the hand as a command that activates a menu for modifying settings and/or for executing operating commands.

24. The auxiliary apparatus according to claim 23, wherein said menu comprises virtual pushbuttons, at least one of which is associated with a specific component of said virtual three-dimensional model, and said processing unit is programmed to activate preset functions on said specific component by virtual pressing of the respective virtual pushbutton.

25. The auxiliary apparatus according to claim 24, wherein said processing unit is programmed to highlight an edge of said specific component when said image of the hand approaches the respective virtual pushbutton (PB).

26. The auxiliary apparatus according to claim 25, wherein said processing unit is programmed to represent a caption which bears a name of said specific component when said hand image approaches the respective virtual pushbutton.

27. The auxiliary apparatus according to claim 19, wherein said processing unit is provided with a video card adapted to receive a video stream of a surgical video camera and is programmed to reproduce on said monitor said video stream in background.

28. The auxiliary apparatus according to claim 19, wherein said structure consists of a cart mounted on casters.

29. The auxiliary apparatus according to claim 28, wherein said cart comprises a post which rises from a base.

30. The auxiliary apparatus according to claim 19, wherein said monitor is connected to said structure by means of a first articulated arm.

31. The auxiliary apparatus according to claim 19, wherein said contactless sensor is connected to said structure by means of a second articulated arm.

32. The auxiliary apparatus according to claim 31, wherein said articulated arm to which the contactless sensor is connected has a substantially horizontal proximal portion and a distal portion which is connected to the substantially horizontal proximal portion by means of a two-axis joint.

33. The auxiliary apparatus according to claim 31, wherein said processing unit is supported on a first fixed shelf which is connected at an intermediate height of the structure on a side that is opposite with respect to said articulated arm to which the contactless sensor is connected.

34. The auxiliary apparatus according to claim 33, further comprising a keyboard which is functionally connected to said processing unit and is supported on an extractable shelf which is fixed to said structure above said first fixed shelf on the opposite side with respect to said articulated arm to which the contactless sensor is connected.

35. The auxiliary apparatus according to claim 34, wherein said extractable shelf is provided with at least one platform which can be extracted laterally for a mouse.

36. The auxiliary apparatus according to claim 31, further comprising counterweights which are fixed to said structure on an opposite side with respect to said second articulated arm to which the contactless sensor is connected.
